(19) United States
(12) Patent Application Publication
(10) Pub. No.: US 2024/0228422 A1
(43) Pub. Date: Jul. 11, 2024

(54) PROCESS FOR PREPARING OXOBUTANOL ESTERS OF POLYMERIC CARBOXYLIC ACIDS

(71) Applicant: IOI Oleo GmbH, Witten (DE)

(72) Inventors: Dirk LOCHMANN, Witten (DE); Sebastian REYER, Witten (DE); Michael STEHR, Witten (DE)

(21) Appl. No.: 18/004,750

(22) PCT Filed: Sep. 7, 2020
(86) PCT No.: PCT/EP2020/074891

§ 371 Date: Jan. 9, 2023

(30) Foreign Application Priority Data
Jul. 13, 2020 (WO) PCT/EP2020/069709

(51) Int. Cl. C07C 67/08; A61K 31/765; C08G 63/91; C12P 7/625

(52) U.S. Cl. CPC C07C 67/08; A61K 31/765; C08G 63/912; C12P 7/625

(57) ABSTRACT

The invention relates to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, as well as the products thus obtained and their use.

### CROSS-REFERENCES TO RELATED APPLICATIONS

This application is a National Stage filing of International Application PCT/EP 2020/074891 filed Sep. 7, 2020, entitled “Process for Preparing Oxobutanol Esters of Polymeric Carboxylic Acids” claiming priority to PCT/EP 2020/069709 filed Jul. 13, 2020. The subject application claims priority to PCT/EP 2020/074891 and PCT/EP 2020/069709 and incorporates all by reference herein, in their entirety.

### BACKGROUND OF THE INVENTION

The present invention relates to the field of keto bodies and related metabolism and the therapy of related diseases.

Especially, the present invention relates to a method for producing oxobutanol esters of polymeric carboxylic acids, as well as the reaction products thus obtainable or thus prepared (i.e. oxobutanol esters of polymeric carboxylic acids) and their use, especially in pharmaceutical compositions, such as drugs or medicaments, or in food and/or food products, as well as their further applications or uses.

Furthermore, the present invention relates to pharmaceutical compositions, especially drugs or medicaments, comprising the reaction products (i.e. oxobutanol esters of polymeric carboxylic acids) obtainable or produced according to the inventive method, as well as their applications or uses.

Finally, the present invention relates to food and/or food products, especially food supplements, functional foods, novel foods, food additives, food supplements, dietary foods, power snacks, appetite suppressants and strength and/or endurance sports supplements, which comprise the reaction products (i.e. oxobutanol esters of polymeric carboxylic acids) obtainable or produced according to the inventive method, as well as their applications or uses.

In the human energy metabolism, glucose is the short-term available energy carrier, which is metabolized into energy in the mitochondria by releasing water and carbon dioxide. The glycogen stores of the liver are already emptied during the sleep period during the night. However, especially the human central nervous system (CNS) and the heart require a permanent energy supply.

The physiological alternative to glucose, which is mainly available to the central nervous system, are the so-called keto bodies (synonymously also called ketone bodies).

The term keto body is especially a collective term for three compounds, which are formed mainly in catabolic metabolic states (such as hunger, reduction diets or low-carbohydrate diets) and may lead to ketosis. The term keto bodies includes especially the three compounds acetoacetate (synonymously also referred to as acetacetate) and acetone as well as 3-hydroxybutyric acid (hereinafter also synonymously referred to as beta-hydroxybutyric acid or BHB or 3-BHB) or its salt (i.e. 3-hydroxybutyrate or beta-hydroxybutyrate), the latter being the most important of the three afore-mentioned compounds. 3-Hydroxybutyric acid or its salt occurs physiologically as the (R)-enantiomer, i.e. as (R)-3-hydroxybutyric acid (synonymously also called (3R)-3-hydroxybutyric acid to emphasize the center of chirality in the 3-position) or its salt.

These keto bodies are also provided physiologically in large amounts from lipids stored in the body by lipolysis during fasting or starvation and replace the energy source glucose almost completely.

The keto bodies are formed in the liver from acetyl coenzyme A (=acetyl-CoA), which originates from beta-oxidation; they represent a transportable form of the acetyl coenzyme A in the human body. However, in order to utilize the keto bodies, the brain and muscles must first adapt by expressing enzymes that are required to convert keto bodies back into acetyl coenzyme A. Especially in times of hunger, the keto bodies contribute a considerable amount to energy production. For example, after some time the brain is able to get by with only a third of the daily amount of glucose.

Physiologically, the keto bodies are synthesized from two molecules of activated acetic acid in the form of acetyl coenzyme A, the normal intermediate product of fatty acid degradation, which is extended using a further acetyl coenzyme A unit and the enzyme HMG-COA-synthase to the intermediate product 3-hydroxy-3-methyl-glutaryl-CoA (HMG-COA), wherein finally the HMG-CoA-lyase cleaves off the acetoacetate. These three steps take place exclusively in the mitochondria of the liver (lynen cycle), wherein 3-hydroxybutyrate is finally formed in the cytosol by the D-beta-hydroxybutyrate dehydrogenase. HMG-CoA is also an end product of the degradation of the amino acid leucine, while acetoacetate is formed during the degradation of the amino acids phenylalanine and tyrosine.

Spontaneous decarboxylation turns acetoacetate into acetone; it can occasionally be perceived in the breath of diabetics and dieters. It cannot be further used by the body. However, the proportion of acetone in the keto bodies is small.

Acetoacetate is thus reductively converted into the physiologically relevant form of 3-hydroxybutyric acid or 3-hydroxybutyrate, but can also decompose into the physiologically unusable acetone with the release of carbon dioxide, which is detectable and olfactory perceptible in severe ketosis, a ketoacidosis (e.g. in diabetes mellitus type 1 patients without insulin substitution), in the urine and in the exhaled air.

3-Hydroxybutyric acid is currently used and marketed in the weight training sector as a sodium, magnesium or calcium salt.

However, 3-hydroxybutyric acid is not known or only in very small quantities to humans in evolutionary terms, since plants do not produce 3-hydroxybutyric acid and 3-hydroxybutyric acid in the animal organism only occurs in dead emaciated animals in ketosis, so that 3-hydroxybutyric acid causes nausea when administered orally. 3-Hydroxybutyric acid in the form of free acid and its salts also taste very bitter and can cause severe vomiting and nausea.

Moreover, patients, especially newborns, but also adults cannot permanently tolerate large amounts of salts of 3-hydroxybutyric acid, as these compounds can have a kidney-damaging effect.

In addition, the plasma half-life of 3-hydroxybutyric acid and its salts is so short that even if several grams are taken, the ketosis lasts only for about three to four hours, i.e. patients cannot benefit continuously from a therapy with 3-hydroxybutyric acid or its salts, especially at night. In case of metabolic diseases this can lead to life-threatening situations.

Therefore, in the case of the therapy of such metabolic diseases, so-called medium-chain triglycerides, so-called MCTs, are currently used for ketogenic therapy, i.e. the metabolic conversion of caproic, caprylic and capric acid (i.e. of saturated linear C₆-, C₈- and C₁₀-fatty acids) from the corresponding triglycerides is intended.

Basically, however, from a pharmaceutical and clinical point of view, 3-hydroxybutyric acid is a more effective pharmaceutical-pharmacological target molecule, which, according to the prior art, could in principle be used for the therapy of a large number of diseases, but cannot be used due to its lack of physiological compatibility (e.g. in diseases in connection with a malfunction of the energy metabolism, especially keto-body metabolism, or neurodegenerative diseases such as dementia, Alzheimer's disease, Parkinson's disease, etc., lipometabolic diseases etc.).

The following table illustrates purely exemplary, but by no means limiting, potential therapy options or possible indications for the active ingredient 3-hydroxybutyric acid.

| Indication | Therapeutic effect |
|---|---|
| Traumatic brain injury | Under BHB the apoptosis and necrosis |
| | rate of nerve cells decreases. |
| Stroke | Under BHB the apoptosis and necrosis |
| | rate of nerve cells decreases. |
| Refeeding syndrome | In case of anorexia, discontinuation of |
| | enteral or parenteral nutrition and after |
| | long periods of hunger, the consumption of |
| | starch or glucose can lead to death (see |
| | also WHO scheme peanut paste). BHB can be |
| | used here as a therapeutic agent to |
| | achieve normal food intake more quickly. |
| Appetite suppressant | BHB suppresses the feeling of hunger |
| | in the central nervous system (CNS). |
| Epilepsy | Conventional ketogenic diet to significantly |
| | reduce the frequency of seizures has extremely |
| | poor patient tolerance. BHB offers an |
| | immediately effective alternative here. |
| Alzheimer's disease, | Under BHB patients show better cognitive |
| dementia | performance. BHB is also effective in the |
| | prevention of neurodegenerative diseases. |
| Disorders of fatty acid | Compensation of a nutrient deficiency in |
| oxidation (e.g. electron | case of defect in energy metabolism. |
| transfer protein defect) | |

Therefore, it is desirable from a pharmaceutical and clinical point of view to be able to find effective precursors or metabolites which physiologically allow direct or indirect access to 3-hydroxybutyric acid or its salts, especially in the physiological metabolism of the human or animal body.

Consequently, the prior art has not lacked attempts to find physiologically suitable precursors or metabolites for 3-hydroxybutyric acid or its salts. So far, however, no efficient compounds have been found in the prior art. Also, access to such compounds is not or not readily possible according to the prior art.

### BRIEF SUMMARY OF THE INVENTION

The problem underlying the present invention is thus the provision of an efficient method for producing physiologically suitable or physiologically compatible precursors and/or metabolites of 3-hydroxybutyric acid (i.e. beta-hydroxybutyric acid or BHB or 3-BHB) or their salts.

Such method should especially make the respective BHB precursors and/or BHB metabolites accessible in an efficient way, especially in larger quantities and without significant amounts of toxic by-products.

In a completely surprising way, the applicant has now discovered that oxobutanol esters of polymeric carboxylic acids represent an efficient and physiologically effective or physiologically compatible precursor and/or metabolite for the keto body 3-hydroxybutyric acid or its salts and has in this context been able to find or develop an efficient method for producing these compounds, which allows direct and effective, especially economic as well as industrially feasible access to these compounds.

To solve the problem described above, the present invention therefore proposes—according to a first aspect of the present invention—a method for producing oxobutanol esters of polymeric carboxylic acids according to the teaching herein; further, especially special and/or advantageous embodiments of the inventive method are the subject-matter of the relevant claims.

Furthermore, the present invention relates—according to as e con d aspect of the present invention—to a reaction product obtainable according to the inventive method disclosed or an oxobutanol ester of a polymeric carboxylic acid or a mixture of at least two oxobutanol esters of polymeric carboxylic acids.

Likewise, the present invention—according to a third aspect of the present invention—relates to a pharmaceutical composition, especially a drug or medicament, especially special and/or advantageous embodiments of this aspect of the invention.

Furthermore, the present invention—according to a fourth aspect of the present invention—relates to an inventive reaction product or an inventive oxobutanol ester of a polymeric carboxylic acid or an inventive mixture of at least two oxobutanol esters of polymeric carboxylic acids for the prophylactic and/or therapeutic treatment or for use in the prophylactic and/or therapeutic treatment of diseases of the human or animal body.

Furthermore, the present invention—according to a fifth aspect of the present invention—relates to the use of an inventive reaction product or an inventive oxobutanol ester of a polymeric carboxylic acid according to the invention or of an inventive mixture of at least two oxobutanol esters of polymeric carboxylic acids for the prophylactic and/or therapeutic treatment or for producing a medicament for the prophylactic and/or therapeutic treatment of diseases of the human or animal body.

Furthermore, the present invention—according to a sixth aspect of the present invention—relates to the use of an inventive reaction product or an inventive oxobutanol ester of a polymeric carboxylic acid or an inventive mixture of at least two oxobutanol esters of polymeric carboxylic acids.

Furthermore, the present invention—according to a seventh aspect of the present invention—relates to a food and/or food product, especially special and/or advantageous embodiments of the food and/or food product.

Finally, the present invention—according to an eighth aspect of the present invention—relates to the use of an inventive reaction product or an inventive oxobutanol ester of a polymeric carboxylic acid or of an inventive mixture of at least two oxobutanol esters of polymeric carboxylic acids in a food and/or a food product.

It goes without saying that following features, embodiments, advantages and the like, which are subsequently listed below only with regard to one aspect of the invention for the purpose of avoiding repetition, naturally also apply accordingly to the other aspects of the invention, without this requiring a separate mention.

Furthermore, it goes without saying that individual aspects and embodiments of the present invention are also considered disclosed in any combination with other aspects and embodiments of the present invention and, especially, any combination of features and embodiments, as it results from back references of all patent claims, is also considered extensively disclosed with regard to all resulting combination possibilities.

With respect to all relative or percentage weight-based data provided below, especially relative quantity or weight data, it should further be noted that within the scope of the present invention these are to be selected by the person skilled in the art such that they always add up to 100% or 100% by weight, respectively, including all components or ingredients, especially as defined below; however, this is self-evident for the person skilled in the art.

In addition, the skilled person may, if necessary, deviate from the following range specifications without leaving the scope of the present invention.

Additionally, it applies that all values or parameters or the like specified in the following can be determined or identified in principle with standardized or explicitly specified determination methods or otherwise with the determination or measurement methods that are otherwise familiar to a person skilled in the art.

Having stated this, the present invention will be described in more detail hereinafter:

### DETAILED DESCRIPTION OF THE INVENTION

The subject-matter of the present invention—according to a first aspect of the present invention—is thus a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

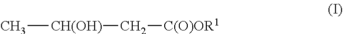

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one polymeric, especially polycondensed or polymerized, carboxylic acid (II) comprising at least three carboxyl groups, especially in an esterification reaction and/or under esterification conditions,
so that, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), is/are obtained.

According to the present invention, there is thus provided a production method for oxobutanol esters, especially 3-hydroxybutanoate esters, of polymeric carboxylic acids. 3-Hydroxybutanoate may also be referred to synonymously as 3-hydroxybutyric acid ester or 4-oxo-2-butanol.

In this context, polymeric carboxylic acids are polymers based on carboxylic acids (i.e. polymers formed by polycondensation or polymerization of carboxylic acids). Polymeric carboxylic acids should not be confused with polycarboxylic acids, which are molecules with more than two carboxyl groups. Especially, polymeric carboxylic acids have at least two repeating units derived from or based on a monomeric carboxylic acid. In this context, “derived from” means that the repeating unit is formed from the monomeric carboxylic acid, especially by (poly)condensation (e.g. esterification) with further identical monomeric carboxylic acids; i.e. the repeating unit is, for example, the radical (dicarboxylate radical) of the corresponding monomeric carboxylic acid, especially esterified twice.

Strictly speaking, in the context of the present invention, oxobutanol (especially also referred to as 3-hydroxybutanoate) of the general formula (I) is either a (C₁-C₅-alkyl)-3-hydroxybutanoate (=3-hydroxybutyric acid (C₁-C₅-alkyl) ester), i.e. a C₁-C₅-alkyl ester of 3-hydroxybutyric acid, which may also be referred to synonymously as a 4-oxo-4-(C₁-C₅-alkoxy)-2-butanol, or else a hydroxy-C₃-C₅-alkyl)-3-hydroxybutanoate (=3-hydroxybutyric acid (hydroxy-C₃-C₅-alkyl) ester), i.e. a hydroxy-C₃-C₅-alkyl ester of 3-hydroxybutyric acid, which can also be referred to synonymously as a 4-oxo-4-(hydroxy-C₃-C₅-alkoxy)-2-butanol.

In this context, the 3-hydroxybutanoates can be prepared by esterification of the free 3-hydroxybutyric acid with the corresponding alcohol (e.g. monoalcohol or diol). In the following, the synthesis of hydroxybutyl 3-hydroxybutanoate (i.e. 3-hydroxybutanoate of general formula (I) with R¹=hydroxybutyl) is shown schematically:

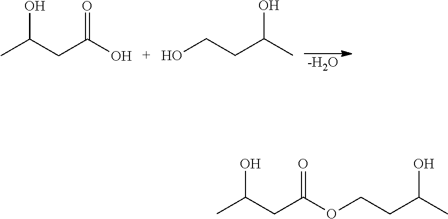

Furthermore, the synthesis of hydroxypentyl 3-hydroxybutanoate (i.e. 3-hydroxybutanoate of the general formula (I) with R¹=hydroxypentyl) is shown schematically below:

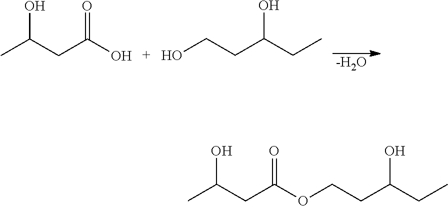

The production or synthesis of further 3-hydroxybutanoates proceeds analogously.

In the method according to the invention, the starting material or compound oxobutanol or 3-hydroxybutanoate of the general formula (I) acts as an esterification alcohol via the hydroxyl group and reacts here with a carboxyl group of the polymeric carboxylic acid (II), so that, as a reaction product (III), a corresponding oxobutanol ester of the polymeric carboxylic acid, especially a 4-oxo-4-(C₁-C₅-alkoxy)-2-butanol ester or a 4-oxo-4-(hydroxy-C₃-C₅-alkoxy)-2-butanol ester of the polymeric carboxylic acid (II) is formed.

In the context of the present invention, both the polymeric carboxylic acid (Il) used and the monomeric carboxylic acid underlying the polymeric carboxylic acid (II) are organic carboxylic acids (i.e. organic compounds having one or more carboxyl groups (—COOH), which have an acidic character).

The use of polymeric carboxylic acids can provide a molecule with high drug density, especially density of 3-hydroxybutanoates or 3-BHB or corresponding derivatives.

In addition, if desired, the products (i.e. oxobutanol esters of polymeric carboxylic acids) can be provided in solid form by using appropriate reactants and adjusting the reaction conditions. A solid dosage form may be advantageous for use in or as drugs, medicaments, or food and/or food products. Also, oxobutanol esters of polymeric carboxylic acids—compared to corresponding oxobutanol esters of monomeric carboxylic acids—exhibit a different solubility, which can be influenced by the specific selection of reactants and reaction conditions.

Surprisingly, the applicant has found a way to provide 3-hydroxybutyric acid or its derivative (i.e. ester) in an organoleptically compatible form, while still allowing the 3-hydroxybutyric acid or its ester to be readily released per se, especially by the animal or human body.

In addition, the applicant has succeeded in providing the organoleptically compatible form of 3-hydroxybutyric acid such that a retardation effect is present, i.e. the 3-hydroxybutyric acid is released continuously over a longer period of time.

In addition, the other degradation or cleavage products (i.e. the cleavage products that are released in addition to 3-hydroxybutyric acid) can also be utilized by the body, or at least processed by the body. Especially, in addition to 3-BHB, degradation or cleavage products are released which are reactants, products or intermediates of the citrate cycle or which are derivatives or salts formed by oxidation of a reactant, product or intermediate of the citrate cycle. Thus, the further degradation or cleavage products formed during the release of 3-hydroxybutyric acid can also be used as an energy source by the animal or human body.

By using polymeric carboxylic acids, a large number of free carboxyl groups are present, so that it is possible to have several oxobutanols, or 3-hydroxybutanoates, react with the polymeric carboxylic acid (i.e. several oxobutanols or 3-hydroxybutanoates are added to a polymeric carboxylic acid), so that a high density of 3-BHB, or its ester, is present within a molecule, resulting in a high drug density.

As stated above, the applicant has, quite surprisingly, discovered that the oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, thus produced are efficient, since physiologically compatible precursors and/or metabolites of 3-hydroxybutyric acid or their salts, which can also be used in larger quantities in pharmaceutical or clinical applications because they are physiologically compatible.

The above-mentioned oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, which are accessible for the first time in an efficient manner through the production method according to the invention, represent a physiologically and pharmacologically relevant alternative to free 3-hydroxybutyric acid or its salts or esters.

The production of oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, by means of conventional organic synthesis is complex and costly, since 3-hydroxybutyric acid has an increased tendency to polymerize and to undergo other undesirable side reactions (e.g. dehydration, decomposition, etc.). Within the scope of the present invention, it was possible for the first time to provide an efficiently working production method with which oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, can be produced without undesired side reactions, especially in a single step or in a one-pot synthesis.

The inventive method thus makes it possible for the first time to provide non-toxic oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, from known, commercially available and above all physiologically harmless components or reactants (starting compounds) as part of a less complex production method. The resulting oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, can be broken down physiologically, especially in the stomach and/or bowl, and release or generate the target molecule “3-hydroxybutyric acid” or its salts or esters as active ingredient or active component.

In addition, the aforementioned oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, also comprise an acceptable taste to ensure compatibility even when administered orally in larger quantities over a longer period of time (e.g. administration of 50 g daily dose or more).

Similarly, the production method according to the invention makes it possible to provide the oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, free from toxic impurities.

In addition, with appropriate starting materials, the method can also be carried out enantioselectively. For example, according to the invention, the production method allows the biologically relevant form, i.e. the (R)-enantiomer of 3-BHB, to be enriched, for example by enzyme catalysis or the targeted selection of the used starting compounds (reactants), as not to burden the renal system of patients when administered orally (i.e. elimination via the kidneys). In principle, however, it is also possible, and under certain conditions may be useful, to enrich the (S)-enantiomer of 3-BHB.

Furthermore, the production method according to the invention, including optional further processing or purification steps, can be operated economically and can also be implemented on a large scale.

Especially, the inventive production method uses commercially available starting compounds or starting materials, which can be synthesized easily and feasibly on a large scale and furthermore allows a relatively simple process management overall even in case of large-scale implementation.

In contrast to conventional prior art production methods, the production method according to the invention does not use complex starting materials and particularly uses only a single step. Nevertheless, excellent yields are achieved in accordance with the invention, wherein the formation of by-products is minimized or avoided.

In addition, the inventive method is simple and economical. Especially, the method according to the invention is usually carried out in the absence of solvents and/or without any solvent (i.e. as a reaction in mass or as a reaction in substance or as a so-called bulk reaction); consequently, the reaction products obtained are not contaminated with solvent and no solvent has to be removed and disposed of or recycled in a costly and energy-intensive manner after the method or reaction has been carried out. Furthermore, no toxic by-products are formed.

The production method according to the invention usually results in a mixture of different oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, i.e. in a mixture of at least two, especially three, different oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids. The resulting raw reaction product or raw mixture can be purified by known methods, especially by removing any remaining starting compounds and/or any by-products present, and furthermore—if desired—can be separated by known methods, especially by distillation and/or chromatography (e.g. fractionation into the individual oxobutanol esters of polymeric carboxylic acids, i.e. separation of the respective monoesters, diesters etc., or else fractionation into fractions with enriched and depleted portions of individuals esters etc.).

As previously stated, the present invention according to the first aspect thus relates to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

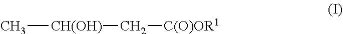

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one polymeric, especially polycondensed or polymerized, carboxylic acid (II) comprising at least three carboxyl groups, especially in an esterification reaction and/or under esterification conditions,
so that, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), is/are obtained.

According to a particular embodiment of the present invention, the compound of the general formula (I) can be used in racemic form or in the form of the (R)-enantiomer. The (R)-configuration refers to the chiral carbon atom in the 3-position of the compound of the general formula (I).

According to the invention, it is preferred when, in the general formula (I), the radical R¹ represents ethyl.

In other words, according to the invention, it is preferred that, as a compound of the general formula (I), 3-hydroxybutyric acid ethyl ester (synonymously also referred to as 3-hydroxybutyric acid ethyl ester or 4-ethoxy-4-oxo-2-butanol) of the formula CH₃—CH(OH)—CH₂—C(O)OC₂H₅ is used.

This enables particularly efficient process control and high yields with minimized or suppressed by-product formation. In addition, the 3-hydroxybutyric acid ethyl ester or 4-ethoxy-4-oxo-2-butanol is also commercially available in large quantities and can especially be obtained on a large scale as a starting compound, e.g. by Claisen condensation of ethyl acetate.

According to a particular embodiment of the present invention, the polymeric carboxylic acid (II) may be used in the form of the free carboxylic acid, in the form of a (partial) salt of the polymeric carboxylic acid or in the form of a (partial) ester of the polymeric carboxylic acid, especially in the form of the free polymeric carboxylic acid.

In the context of the present invention, it may be provided that the polymeric carboxylic acid (II) is produced by polycondensation or by polymerization, especially ring-opening polymerization.

In the production by polycondensation, for example, the polymeric carboxylic acid is esterified by an esterification reaction of the hydroxyl group of one carboxylic acid with the carboxyl group of another carboxylic acid. In this context, identical carboxylic acids are esterified with each other, i.e. two molecules of the same carboxylic acid, for example two tartaric acid molecules or two citric acid molecules, are esterified with each other. The reaction method is shown schematically below using tartaric acid as an example:

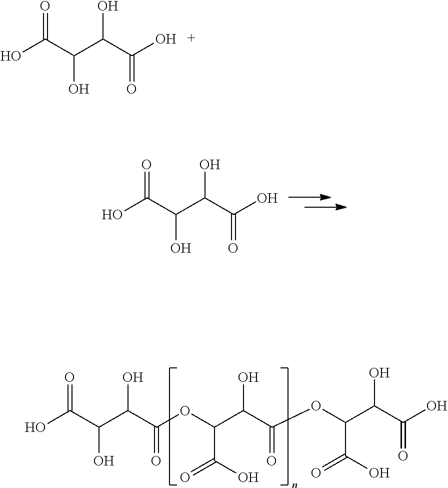

Alternatively, the polymeric carboxylic acid can be prepared by ring-opening polymerization of the lactone of the carboxylic acid. Lactones are heterocyclic compounds which have an ester bond within the ring. The reaction method is shown schematically below using tartaric acid as an example:

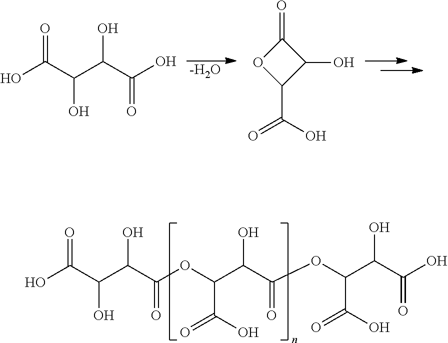

The corresponding reactants for the production of the polymeric carboxylic acid (i.e. the monomeric carboxylic acids or their corresponding lactones) are commercially available, and the polycondensation or ring-opening polymerization is simple and feasible on a large scale. Alternatively, many polymeric carboxylic acids are also commercially available.

In the context of the method according to the invention, it may be preferred if the polymeric carboxylic acid (II) is an ingredient, especially an additive, approved under food law.

Ingredients or additives approved under food law are listed throughout the EU in a list of food additives and are given their own labeling in this context (so-called E-number). For example, metatartaric acid (E353), synonymously also polytartaric acid, is a polymeric carboxylic acid approved under food law.

Furthermore, it may also be preferred if the polymeric carboxylic acid (II) has two terminal and/or primary carboxyl groups. In this context, the terminal and/or primary carboxyl groups are located at the opposite ends of the molecule.

According to a preferred embodiment of the method according to the invention, it may be provided that the polymeric carboxylic acid (II) comprises at least two, especially two to twenty, preferentially two to ten, more preferably three to seven, repeating units derived from the monomeric carboxylic acid underlying the polymeric carboxylic acid (II).

The monomeric carboxylic acid underlying the polymeric carboxylic acid (II) is the carboxylic acid that is polycondensed or polymerized to synthesize the corresponding polymeric carboxylic acid (II). For example, tartaric acid is the underlying monomeric carboxylic acid of polytartaric acid (metatartaric acid), and in the case of polycitric acid, citric acid is the underlying monomeric carboxylic acid.

Especially, the repeating units each comprise at least one free carboxyl group. Due to the at least one free carboxyl group in the repeating unit, a particularly large number of oxobutanols can be esterified with the polymeric carboxylic acid and thus a high active ingredient density can be provided.

Typically, the repeating units and/or the monomeric carboxylic acid underlying the polymeric carboxylic acid (II) are derived from and/or selected from hydroxydi- and -polycarboxylic acids, preferentially derived from and/or selected from the group of tartaric acid, malic acid and citric acid as well as combinations and mixtures thereof, more preferably derived from and/or selected from tartaric acid.

In this context, “derived from” means that the hydroxy and polycarboxylic acids underlie the repeating units and the monomeric carboxylic acid, respectively. In the case of the repeating units, this is formed by esterification of the hydroxydi- and- polycarboxylic acids and it is not the hydroxydi- and -polycarboxylic acids per se that form the repeating units, but their (di)carboxylate. Hydroxydi- and -polycarboxylic acids refer to organic substances that have at least one hydroxyl group and at least two carboxyl groups.

These carboxylic acids are particularly suitable for conversion to polymeric carboxylic acids and are also commercially available and, moreover, additives or ingredients approved for use in foodstuffs.

Usually, the monomeric carboxylic acid on which the polymeric carboxylic acid (II) is based is a naturally occurring carboxylic acid or derivative thereof, especially a reaction product, especially a carboxylic acid or derivative thereof, especially a reaction product, occurring in human and/or animal metabolism.

Polymeric carboxylic acids which are based on carboxylic acids or derivatives which are part of the human and/or animal metabolism or are reactants or products or intermediates of a human and/or animal metabolism are particularly compatible in the use of the reaction product obtained in or as a drug, medicament or food and/or food product. Especially, it is advantageous in this context if, the monomeric carboxylic acids on which the polymeric carboxylic acid or derivatives thereof are based, are those which occur in the citrate cycle, result from the citrate cycle or are associated with the citrate cycle. In this context, derivatives can represent, for example, salts or esters that are obtainable by oxidation of a metabolic product (for example, from the citrate cycle). By using polymeric carboxylic acids which are based on monomeric carboxylic acids or derivatives which are part of the human and/or animal metabolism or are reactants or products or intermediates of a human and/or animal metabolism, a further energy source (in addition to the keto body 3-hydroxybutyric acid or 3-hydroxybutanoate) can be supplied or made available to the human and/or animal body when using the reaction product according to the invention.

Especially, the monomeric carboxylic acid on which the polymeric carboxylic acid (II) is based is an ingredient, especially an additive, approved under food law.

For example, the following carboxylic acids are listed in the food additive list and are also suitable for the preparation of a polymeric carboxylic acid (II) that can be used in the method according to the invention: tartaric acid (E334), citric acid (E330) and malic acid (E296). These acids are part of the citrate cycle or are obtainable by oxidation of a metabolic product of the citrate cycle. The citrate cycle is a cycle of biochemical reactions which plays an important role in the metabolism of aerobic cells of living organisms and is mainly used for the oxidative degradation of organic substances for the purpose of energy production and the provision of intermediates for biosynthesis. Thus, the carboxylic acids formed by degradation when using the reaction product (III) obtainable from the method according to the invention can be utilized by the body as a further alternative energy source.

According to a particular embodiment of the method of the invention, the polymeric carboxylic acid (II) may be selected from polymeric hydroxydi- and -polycarboxylic acids, preferentially from the group of polytartaric acid (metatartaric acid or E 353), polymalic acid and polycitric acid as well as their (partial) salts and (partial) esters and combinations and mixtures thereof, more preferably polytartaric acid (metatartaric acid or E 353) as well as their (partial) salts and (partial) esters and mixtures thereof, even more preferably polytartaric acid (metatartaric acid or E 353).

In this context, polytartaric acid or its salts is also known as polytartate, polymalic acid or its salts is also known as polymalate, and polycitric acid or its salts is also known as polycitrate.

According to a particular embodiment of the present invention, it is preferred if the polymeric carboxylic acid (II) is selected from polytartaric acid (metatartaric acid or E 353) as well as its (partial) salts and (partial) esters and mixtures thereof, especially polytartaric acid (metatartaric acid or E 353).

Polytartaric acid, synonymously also called metatartaric acid, is particularly suitable because it is an additive approved under food law (E-number: E 353) and is therefore particularly suitable for the production of a product used as a drug, medicine or food and/or food product.

According to a particular embodiment of the present invention, the polymeric carboxylic acid (II) may correspond to the general formula (IIa)

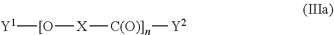

wherein, in the general formula (IIa),
   X represents: an organic radical, especially a saturated or unsaturated, linear or branched organic radical comprising from 3 to 10, preferentially from 3 to 8, carbon atoms and comprising from 1 to 3 carboxyl groups, and optionally comprising at least one hydroxyl group;
   the variable n represents an (integer) number≥2, especially in the range of from two to twenty, preferentially in the range of from two to ten, more preferably in the range of from three to seven;
   Y¹ and Y², each independently of one another, represent a saturated or unsaturated, linear or branched organic radical comprising a terminal and/or primary carboxyl group and comprising three to seven carbon atoms and four to eight oxygen atoms.

According to another particular embodiment of the present invention, the polymeric carboxylic acid (II) may correspond to the general formula (IIb)

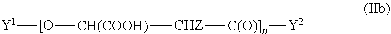

wherein, in the general formula (IIb),
   Z represents hydrogen or a hydroxyl group;
   the variable n represents an (integer) number≥2, especially in the range of from two to twenty, preferentially in the range of from two to ten, more preferably in the range of from three to seven;
   Y¹ represents a group HOOC—CH(OH)—CH₂—C(O)— in case Z is hydrogen, or represents a group HOOC—CH(OH)—CH(OH)—C(O)— in case Z is a hydroxyl group;
   Y² represents a group —O—CH(COOH)—CH₂—COOH in case Z is hydrogen, or represents a group —O—CH(COOH)—CH(OH)—COOH in case Z is a hydroxyl group.

According to yet another particular embodiment of the present invention, the polymeric carboxylic acid (II) may correspond to the general formula (IIc)

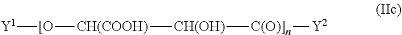

wherein, in the general formula (IIc),
   the variable n represents an (integer) number≥2, especially in the range of from two to twenty, preferentially in the range of from two to ten, more preferably in the range of from three to seven;
   Y¹ represents a group HOOC—CH(OH)—CH(OH)—C(O)—;
   Y² represents a group —O—CH(COOH)—CH(OH)—COOH.

According to a particular embodiment of the present invention, the polymeric carboxylic acid (II) may correspond to the general formula (IId)

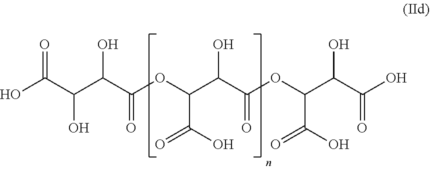

wherein, in the general formula (IId), the variable n represents an (integer) number≥2, especially in the range of from two to twenty, preferentially in the range of from two to ten, more preferably in the range of from three to seven.

According to a particular embodiment of the present invention, the present invention relates, in accordance with this aspect of the invention, to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)- 2-butanol esters of polymeric carboxylic acids, especially a method as described hereinabove,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

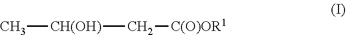

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II) selected from polymeric hydroxydi- and -polycarboxylic acids, preferentially from the group of polytartaric acid (metatartaric acid or E 353), polymalic acid and polycitric acid as well as their (partial) salts and (partial) esters and combinations and mixtures thereof, more preferably from polytartaric acid (metatartaric acid or E 353) as well as their (partial) salts and (partial) esters and mixtures thereof, even more preferably polytartaric acid (metatartaric acid or E 353), especially in an esterification reaction and/or under esterification conditions,
so that, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), are obtained.

According to another particular embodiment of the present invention, the present invention according to this aspect of the invention also relates to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, especially a method as described hereinabove,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

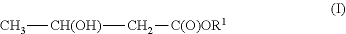

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II) selected from polytartaric acid (metatartaric acid or E 353) as well as its (partial) salts and (partial) esters and mixtures thereof, especially polytartaric acid (metatartaric acid or E 353), especially in an esterification reaction and/or under esterification conditions,
so that, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), are obtained.

According to yet another particular embodiment of the present invention, the present invention according to this aspect of the invention also relates to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, especially a method as described hereinabove,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

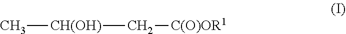

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II) of the general formula (IIc)

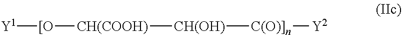

wherein, in the general formula (IIc),
   the variable n represents an (integer) number≥2, especially in the range of from two to twenty, preferentially in the range of from two to ten, more preferably in the range of from three to seven;
   Y¹ represents a group HOOC—CH(OH)—CH(OH)—C(O)—;
   Y² represents a group —O—CH(COOH)—CH(OH)—COOH,
especially in an esterification reaction and/or under esterification conditions,
so that, as a reaction product (III), one or more oxobutanol esters of the polymeric carboxylic acid (IIc), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (IIc), are obtained.

Furthermore, according to a particular embodiment of the present invention according to this aspect of the invention, the present invention also relates to a method for producing oxobutanol esters of polymeric carboxylic acids, especially 4-oxo-4-(C₁-C₅-alkoxy)-2-butanol esters or 4-oxo-4-(hydroxy-C₃-C₅-alkoxy)-2-butanol esters of polymeric carboxylic acids, especially a method as described hereinabove,
wherein at least one oxobutanol, especially at least one 3-hydroxybutanoate, preferentially at least one 4-oxo-4-(C₁-C₅-alkoxy)-2-butanol or 4-oxo-4-(hydroxy-C₃-C₅-alkoxy)-2-butanol, of the general formula (I)

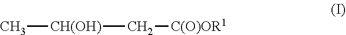

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II) of the general formula (IId)

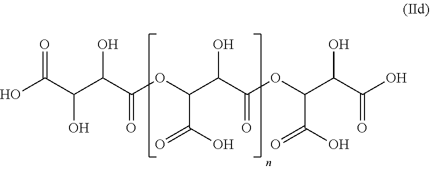

wherein, in the general formula (IId), the variable n represents an (integer) number≥2, especially in the range of from two to twenty, preferentially in the range of from two to ten, more preferably in the range of from three to seven,
especially in an esterification reaction and/or under esterification conditions,
so that, as a reaction product (III), one or more oxobutanol esters of the polymeric carboxylic acid (IId), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (IId), are obtained.

According to a particular embodiment of the present invention, the reaction can be carried out in the absence of solvents and/or without any solvent. This means that the reaction is carried out as a reaction in mass or as a reaction in substance or as a so-called bulk reaction. This has the advantage that the reaction products obtained are not contaminated with solvent and that no solvent has to be removed and disposed of or recycled in a costly and energy-intensive manner after the method or reaction has been carried out. Surprisingly, the method or reaction nevertheless proceeds with high conversion and yields and at least essentially without significant by-product formation.

According to another particular embodiment of the present invention, the reaction may be carried out in the absence of a catalyst and/or without any catalyst, or else the reaction may alternatively be carried out in the presence of a catalyst, especially an enzyme and/or a metal-containing and/or metal-based, acidic or basic catalyst, wherein the catalyst is recycled after the reaction.

In the method according to the invention, it is particularly preferred if the reaction is carried out in the absence of solvents and/or without any solvent and if the reaction is carried out in the absence of a catalyst and/or without any catalyst.

Due to the absence of solvent and the absence of a catalyst, the reaction products are not contaminated by solvent that has not been completely removed or catalyst that has not been completely removed. In addition, the energy-intensive and costly removal or separation steps are eliminated. Surprisingly, the inventive method according to this preferred embodiment is nevertheless economical and results in high conversions without significant by-product formation.

As previously stated, according to a particular embodiment of the production method of the invention, the reaction may be carried out in the absence of a catalyst and/or without any catalyst.

Provided that the reaction is carried out in the absence of a catalyst and/or without any catalyst, it is preferred if the reaction is carried out at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

In case of the reaction in the absence of a catalyst, the applied pressure range can vary within wide ranges. Especially, the reaction can be carried out in the absence of a catalyst and/or without any catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

When reacting in the absence of a catalyst, it is preferred if the reaction is carried out in the presence of an inert gas, especially in the presence of helium, argon or nitrogen, preferably in the presence of nitrogen. Especially, undesirable side reactions, especially due to oxidation or hydrolysis, can be prevented in this way.

Alternatively to this particular embodiment, however, it is also possible to carry out the reaction, for example, in the presence of an enzyme as a catalyst.

In this context, the enzyme can especially be selected from synthetases (ligases), catalases, esterases, lipases and combinations thereof. According to the invention, synthetases (synonymously ligases) are especially enzymes from the class of ligases; ligases are enzymes which catalyze the linking of two or more molecules by a covalent bond. Catalases in the sense of the present invention are especially enzymes which are capable of converting hydrogen peroxide to oxygen and water. The term esterases refers in particular to enzymes which are capable of hydrolytically splitting esters into alcohol and acid (saponification); these are thus especially hydrolases, wherein fat splitting esterases are also called lipases. Lipases in the sense of the present invention are especially enzymes which are capable of splitting free fatty acids from lipids such as glycerides (lipolysis).

In this context, the enzyme used as a catalyst can especially be derived from Candida antarctica, Mucor miehei (Rhizomucor miehei), Thermomyces lanuginosus, Candida rugosa, Aspergillus oryzae, Pseudomonas cepacia, Pseudomonas fluorescens, Rhizopus delemar and Pseudomonas sp. and combinations thereof, preferentially from Candida antarctica, Mucor miehei (Rhizomucor miehei) and Thermomyces lanuginosus.

According to a specific embodiment, the enzyme can be used in immobilized form, especially immobilized on a carrier, preferentially on a polymeric carrier, preferably on a polymeric organic carrier, more preferably with hydrophobic properties, even more preferably on a poly(meth)acrylic resin-based carrier.

In the context of the present invention, it is preferred that, when an enzyme is used as a catalyst, the enzyme is recycled after the reaction.

If the reaction is carried out in the presence of an enzyme as a catalyst within the framework of the inventive production method, it is preferred if the reaction is carried out at temperatures in the range of from 10° C. to 80° C., especially in the range of from 20° C. to 80° C., preferentially in the range of from 25° C. to 75° C., more preferably in the range of from 45° C. to 75° C., even more preferably in the range of from 50° C. to 70° C.

In case of using an enzyme as a catalyst, the amount of the enzyme used can vary within wide ranges. Especially, the enzyme can be used in amounts, based on the total amount of the starting compounds (I) and (II), in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight. Nevertheless, it may be necessary to deviate from the above-mentioned amounts in individual cases or for specific applications without leaving the scope of the present invention.

If, according to a particular embodiment of the present invention, the reaction is carried out in the presence of an enzyme as a catalyst, the applied pressure range may also vary within wide ranges. Typically, the reaction in the presence of an enzyme as a catalyst can be carried out at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 0.5 bar.

According to the particular embodiment of the present invention, according to which the reaction is carried out in the presence of an enzyme as a catalyst, it is preferred if the reaction is carried out in the presence of an enzyme in the presence of an inert gas, especially in the presence of helium, argon or nitrogen, preferably in the presence of nitrogen. As previously stated in the context of the reaction in the absence of a catalyst, undesirable side reactions, especially due to oxidation or hydrolysis, can be prevented by the reaction in the presence of an inert gas.

According to another alternative embodiment of the present invention, the reaction may be carried out, for example, in the presence of a metal-containing and/or metal-based, acidic or basic catalyst.

According to this alternative embodiment of the present invention, according to which the reaction is carried out in the presence of a metal-containing and/or metal-based, acidic or basic catalyst, the catalyst can especially be selected from (i) basic catalysts, especially alkali or alkaline earth hydroxides and alkali or alkaline earth alcoholates, such as NaOH, KOH, LIOH, Ca(OH)₂, NaOMe, KOMe and Na(OBu-tert.), (ii) acidic catalysts, especially mineral acids, and organic acids, such as sulfuric acid, hydrochloric acid, phosphoric acid, nitric acid, sulfonic acids, methane sulfonic acid, para-toluene sulfonic acid and carboxylic acids, (iii) Lewis acids, especially Lewis acids based on titanium, tin, zinc and aluminum compounds, such as titanium tetrabutylate, tin acids, zinc acetate, aluminum tri-chloride and aluminum tri-isopropyl, and (iv) heterogeneous catalysts, especially based on mineral silicates, germanates, carbonates and aluminum oxides, such as zeolites, montmorillonites, mordenites, hydrotalcites and aluminas, and combinations thereof.

In this embodiment, a Lewis acid based on titanium, tin, zinc and aluminum compounds, such as titanium tetrabutylate, tin acids, zinc acetate, aluminum trichloride and aluminum tri-isopropyl, may be used as a catalyst.

Especially, it is also preferred in this embodiment if the metal-containing and/or metal-based acidic or basic catalyst is recycled after the reaction.

If, according to the particular embodiment of the present invention the reaction is carried out in the presence of a metal-containing and/or metal-based, acidic or basic catalyst, the temperatures can be varied within wide ranges. Especially, the reaction can be carried out in the presence of a metal-containing and/or metal-based, acidic or basic catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

Furthermore, also according to this embodiment, the catalyst (i.e. in the presence of a metal-containing and/or metal-based, acidic or basic catalyst) can be varied within wide quantity ranges: Thus, the catalyst can be used in amounts, based on the total amount of starting compounds (I) and (II), in the range of from 0.01% by weight to 30% by weight, especially in the range of from 0.05% by weight to 15% by weight, preferentially in the range of from 0.1% by weight to 15% by weight, preferably in the range of from 0.2% by weight to 10% by weight. Nevertheless, it is possible to deviate from the above-mentioned amounts for specific applications or individual cases without leaving the scope of the present invention.

If, according to this particular embodiment of the present invention, the reaction is carried out in the presence of a metal-containing and/or metal-based, acidic or basic catalyst, the pressure range can equally vary within wide ranges: Especially, the reaction can be carried out in the presence of a metal-containing and/or metal-based, acidic or basic catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Furthermore, also according to this particular embodiment of the present invention, according to which the reaction is carried out in the presence of a metal-containing and/or metal-based, acidic or basic catalyst, the reaction can be carried out in the presence of an inert gas, especially in the presence of helium, argon or nitrogen, preferably in the presence of nitrogen. As previously stated, the reaction in the presence of an inert gas prevents undesirable side reactions, especially due to oxidation or hydrolysis.

As far as the total quantity of starting materials or starting compounds is concerned, this can be varied within wide ranges.

Taking into account process economy and optimization of the course of the method, especially with regard to the minimization of by-products, it is advantageous if the oxobutanol of the general formula (I), based on the carboxyl groups of the polymeric carboxylic acid (II), is used in molar amounts in a range of from equimolar amount up to a molar excess of 200 mol-%, especially in a range of from equimolar amount up to a molar excess of 150 mol-%, preferentially in a range of from equimolar amount up to a molar excess of 100 mol-%.

Similarly, taking into account process economy and optimization of the course of the method, especially with regard to minimizing by-products, it is advantageous if the oxobutanol of the general formula (I) and the polymeric carboxylic acid (II) are used in a molar ratio of oxobutanol of the general formula (I)/carboxyl groups of the polymeric carboxylic acid (II) in a range of from 1:1 to 10:1, especially in a range of from 2:1 to 8:1, preferentially in a range of from 3:1 to 6:1.

In the method according to the invention, during the reaction of oxobutanol of the general formula (I) with a polymeric carboxylic acid (II) in the form of the free acid, water is formed simultaneously. Especially, it is preferred if the water is withdrawn from the reaction, especially continuously withdrawn, especially by means of preferentially continuous, especially distillative or adsorptive removal.

Alternatively, in the method according to the invention, during the reaction of oxobutanol of the general formula (I) with a polymeric carboxylic acid (II) in the form of the ester, one mol of the corresponding alcohol is formed per mol of ester group reacted. Especially, it is preferred if the alcohol is withdrawn from the reaction, especially continuously withdrawn, especially by means of preferentially continuous, especially distillative or adsorptive removal.

In this context, the term “ester group reacted” refers to the ester group which is transesterified in the method according to the invention, i.e. to the ester group which reacts with the oxobutanol. In this context, the corresponding alcohol is split off; i.e. if an ethyl ester of the polymeric carboxylic acid is present, ethanol is split off by the transesterification with an oxobutanol.

Continuous removal of the by-product (i.e. water or alcohol) can especially shift the chemical equilibrium toward the products and thus increase the conversion.

In the production method according to the invention, the composition of the reaction product, especially the presence of the various oxobutanol esters of polymeric carboxylic acids, and their proportion in the case of a mixture, may be controlled and/or regulated by means of the reaction conditions, especially by selecting the reaction temperature (conversion temperature) and/or by selecting the reaction pressure (conversion pressure) and/or absence of or by providing a catalyst and selecting such catalyst with respect to the type and/or amount and/or by selecting the amounts of starting compounds (educts) and/or by providing the removal of the optionally formed by-products, especially water and/or alcohol.

Thus, it is possible to tailor the exact composition of the product depending on the application, especially, for example, the number of substituted oxobutanol radicals (3-hydroxybutanoate radicals) can be adjusted so that the density of keto bodies in the form of oxobutanols or 3-hydroxybutanoates per molecule can be purposefully adjusted.

After the reaction, the reaction product obtained can be subjected to further purification or work-up steps.

In this context, the reaction product obtained can be fractionated after the reaction has been performed, especially fractionated by distillation.

Also, unreacted starting compounds (I) and/or (II) can be separated from the reaction product and subsequently recycled.

According to a special embodiment of the production method according to the invention, it is especially possible to proceed in such a way that hydroxyl groups and/or carboxyl groups still present in the reaction product after the reaction has been performed are at least partially, preferentially completely, functionalized, especially esterified.

Especially, the reaction can be followed by a partial, especially complete functionalization, especially esterification, of hydroxyl groups and/or carboxyl groups still present.

In this particular embodiment of the inventive method, the functionalization, especially esterification, of the hydroxyl groups and/or carboxyl groups still present can be carried out by reaction with a carboxylic acid anhydride of, for example, C₂-C₃₀-carboxylic acids or C₂-C₃₀-fatty acids in case of free hydroxyl groups or C₂-C₃₀-fatty alcohols in case of free carboxyl groups. These may be linear or branched, saturated or mono- or polyunsaturated C₂-C₃₀-carboxylic acid anhydrides or C₂-C₃₀-fatty acid anhydrides or C₂-C₃₀-fatty alcohols. In this context, hydroxyl groups still present can be reacted especially with carboxylic acid anhydrides or fatty acids, and carboxyl groups still present can be reacted especially with fatty alcohols to each result in the corresponding esters.

In the method according to the invention, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), may be obtained.

Especially, in the production method according to the invention, as a reaction product (III), one or more oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), as defined hereinbelow, may be obtained.

According to a particular embodiment of the method of the invention, as a reaction product (III), a mixture of at least two, especially at least three, different oxobutanol esters of the polymeric carboxylic acid (II), especially one or more 4-oxo-4-(C₁-C₅-alkoxy)-2-butanol esters or 4-oxo-4-(hydroxy-C₃-C₅-alkoxy)-2-butanol esters of the polymeric carboxylic acid (II), especially as defined hereinabove and hereinbelow, may be obtained.

A further subject-matter—according to a second aspect of the present invention—is a reaction product, especially a (chemical) product or product mixture, preferentially a oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, or a mixture of several oxobutanol esters of a polymeric carboxylic acid, especially a mixture of several 4-oxo-4-(C₁-C₅-alkoxy)-2-butanol esters or 4-oxo-4-(hydroxy-C₃-C₅-alkoxy)-2-butanol esters of a polymeric carboxylic acid, obtainable by the method described hereinabove.

Especially, the present invention also relates to an oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, especially as defined hereinabove and hereinbelow.

According to a particular embodiment of this aspect of the invention, at least one carboxyl group, especially at least one terminal and/or primary carboxyl group, of the polymeric carboxylic acid may be esterified with an oxobutanol, especially with at least one 3-hydroxybutanoate, preferentially with at least one 4-oxo-4-(C₁-C₅-alkoxy)-2-butanol or 4-oxo-4-(hydroxy-C₃-C₅-alkoxy)-2-butanol, of the general formula (I)

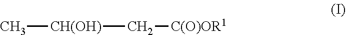

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl.

According to another particular embodiment of this aspect of the invention, the polymeric carboxylic acid may be produced by polycondensation or by polymerization, especially ring-opening polymerization.

In accordance with this aspect of the invention, it may be preferred if the polymeric carboxylic acid is an ingredient, especially an additive, approved under food-law. As previously stated in connection with the method according to the invention, the inventive oxobutanol esters of polymeric carboxylic acids, which contain polymeric carboxylic acids approved under food law, are particularly suitable for use in or as medicaments, drugs, food and/or food products.

Furthermore, according to this aspect of the invention, it may also be preferred if the polymeric carboxylic acid comprises two terminal and/or primary carboxyl groups.

According to a particular embodiment of this aspect of the invention, it may be advantageous if the polymeric carboxylic acid comprises at least two, especially two to twenty, preferentially two to ten, more preferably three to seven, repeating units derived from the monomeric carboxylic acid underlying the polymeric carboxylic acid.

Typically, the repeating units each comprise at least one free carboxyl group. When each repeating unit contains at least one free carboxyl group, it is possible to provide a high drug density since each free carboxyl group can be esterified by or with an oxobutanol.

Especially, the repeating units and/or the monomeric carboxylic acid underlying the polymeric carboxylic acid are derived from and/or selected from hydroxydi- and -polycarboxylic acids, preferentially derived from and/or selected from the group of tartaric acid, malic acid and citric acid as well as combinations and mixtures thereof, more preferably derived from and/or selected from tartaric acid.

As stated previously, “derived from” means that the hydroxydi- and -polycarboxylic acids form the basis of the repeating units or the monomeric carboxylic acid, respectively. In the case of the repeating units, this is formed by esterification of the hydroxydi- and -polycarboxylic acids, and it is not the hydroxydi- and -polycarboxylic acids per se that form the repeating units. Hydroxydi- and -polycarboxylic acids refer to organic substances which have at least one hydroxyl group and at least two carboxyl groups.

Preferably, the monomeric carboxylic acid on which the polymeric carboxylic acid is based is a naturally occurring carboxylic acid or derivative thereof, especially a reaction product, especially a carboxylic acid or derivative thereof, especially a reaction product, occurring in human and/or animal metabolism.

As previously stated, polymeric carboxylic acids which are based on carboxylic acids or derivatives which are part of the human and/or animal metabolism or are reactants or products or intermediates of a human and/or animal metabolism are particularly compatible in the use of the reaction product obtained in or as a drug, medicament or food and/or food product. Especially, it is advantageous in this context if, the monomeric carboxylic acids on which the polymeric carboxylic acid or derivatives thereof are based, are those which occur in the citrate cycle, result from the citrate cycle or are associated with the citrate cycle. In this context, derivatives can represent, for example, salts or esters that are obtainable by oxidation of a metabolic product (for example, from the citrate cycle). By using polymeric carboxylic acids which are based on monomeric carboxylic acids or derivatives which are part of the human and/or animal metabolism or are reactants or products or intermediates of a human and/or animal metabolism, a further energy source (in addition to the keto body 3-hydroxybutyric acid or 3-hydroxybutanoate) can be supplied or made available to the human and/or animal body when using the reaction product according to the invention

Especially, the monomeric carboxylic acid on which the polymeric carboxylic acid is based is an ingredient, especially an additive, approved under food law.

As previously stated, the following carboxylic acids, for example, are listed in the food additive list and are also suitable for the production of a polymeric carboxylic acid (II) that can be used in the method according to the invention: tartaric acid (E334), citric acid (E330) and malic acid (E296). These acids are part of the citrate cycle or are obtainable by oxidation of a metabolic product of the citrate cycle. The citrate cycle is a cycle of biochemical reactions which plays an important role in the metabolism of aerobic cells of living organisms and mainly serves the oxidative degradation of organic substances for the purpose of energy production and the provision of intermediate products for biosynthesis. Thus, the acids formed by degradation when using the reaction product (III) obtainable from the method according to the invention can be utilized by the body as another alternative source of energy.

According to a particular embodiment of this aspect of the invention, the polymeric carboxylic acid may be selected from polymeric hydroxydi- and -polycarboxylic acids, preferentially from the group of polytartaric acid (metatartaric acid or E 353), polymalic acid and polycitric acid as well as their (partial) salts and (partial) esters and combinations and mixtures thereof, more preferably polytartaric acid (metatartaric acid or E 353) as well as their (partial) salts and (partial) esters and mixtures thereof, even more preferably polytartaric acid (metatartaric acid or E 353).

According to a preferred embodiment of this aspect of the invention, the polymeric carboxylic acid may be selected from polytartaric acid (metatartaric acid or E 353) as well as its (partial) salts and (partial) esters and mixtures thereof, especially polytartaric acid (metatartaric acid or E 353).

According to another particular embodiment of the present aspect of the invention, the polymeric carboxylic acid may correspond to the general formula (IIa)

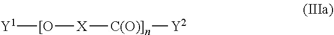

wherein, in the general formula (IIa),
   X represents: an organic radical, especially a saturated or unsaturated, linear or branched organic radical comprising from 3 to 10, preferentially from 3 to 8, carbon atoms and comprising from 1 to 3 carboxyl groups, and optionally comprising at least one hydroxyl group;
   the variable n represents an (integer) number≥2, especially in the range of from two to twenty, preferentially in the range of from two to ten, more preferably in the range of from three to seven;
   Y¹ and Y², each independently of one another, represent a saturated or unsaturated, linear or branched organic radical comprising a terminal and/or primary carboxyl group and comprising three to seven carbon atoms and four to eight oxygen atoms.

According to yet another particular embodiment of the present aspect of the invention, the polymeric carboxylic acid may correspond to the general formula (IIb)

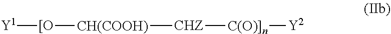

wherein, in the general formula (IIb),
   Z represents hydrogen or a hydroxyl group;
   the variable n represents an (integer) number≥2, especially in the range of from two to twenty, preferentially in the range of from two to ten, more preferably in the range of from three to seven;
   Y¹ represents a group HOOC—CH(OH)—CH₂—C(O)— in case Z is hydrogen, or represents a group HOOC—CH(OH)—CH(OH)—C(O)— in case Z is a hydroxyl group;
   Y² represents a group —O—CH(COOH)—CH₂—COOH in case Z is hydrogen, or represents a group —O—CH(COOH)—CH(OH)—COOH in case Z is a hydroxyl group.

According to still another particular embodiment of this aspect of the invention, the polymeric carboxylic acid may correspond to the general formula (IIc)

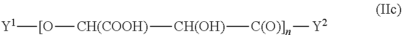

wherein, in the general formula (IIc),
   the variable n represents an (integer) number≥2, especially in the range of from two to twenty, preferentially in the range of from two to ten, more preferably in the range of from three to seven;
   Y¹ represents a group HOOC—CH(OH)—CH(OH)—C(O)—;
   Y² represents a group —O—CH(COOH)—CH(OH)—COOH.

Furthermore, according to a particular embodiment of this aspect of the invention, the polymeric carboxylic acid may correspond to the general formula (IId)

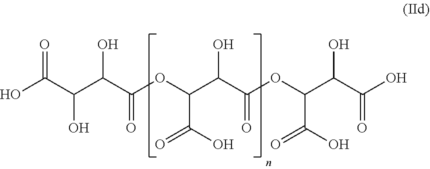

wherein, in the general formula (IId), the variable n represents an (integer) number≥2, especially in the range of from two to twenty, preferentially in the range of from two to ten, more preferably in the range of from three to seven.

It is also an object of the present invention according to a particular embodiment to provide an oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, especially an oxobutanol ester of a polymeric carboxylic acid as defined hereinabove,
wherein the oxobutanol ester corresponds to the general formula (IIIa)

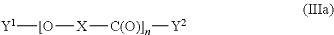

wherein, in the general formula (IIIa),
   X represents: an organic radical, especially a saturated or unsaturated, linear or branched organic radical comprising from 3 to 10, especially from 3 to 8 carbon atoms and comprising from 1 to 3 groups —COOR³ and optionally comprising at least one hydroxyl group, wherein the radical R³ represents, independently of one another with respect to the respective repeating unit: hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
   the variable n represents an (integer) number≥2, especially in the range of from two to twenty, preferentially in the range of from two to ten, more preferably in the range of from three to seven;
   Y¹ and Y², each independently of one another, represent: a saturated or unsaturated, linear or branched organic radical comprising a terminal and/or primary group —COOR⁴, which comprises from three to seven carbon atoms and from four to eight oxygen atoms, wherein, in the group —COOR⁴, the radical R⁴ represents: hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
however, with the proviso that at least one, especially at least two, of the radicals R³ and R⁴ represents a radical —CH(CH₃)—CH₂—C(O)OR¹, as defined above, preferentially with the (further) proviso that at least one radical R⁴ represents a radical —CH(CH₃)—CH₂—C(O)OR¹, as defined above.

A further object of the present invention according to a particular embodiment is also an oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, especially an oxobutanol ester of a polymeric carboxylic acid as defined hereinabove,
wherein the oxobutanol ester corresponds to the general formula (IIIb) is

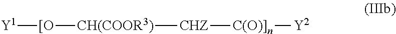

wherein, in the general formula (IIIb),
   Z represents hydrogen or a hydroxyl group;
   R³, independently of one other with respect to the respective repeating unit, represents: hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
   the variable n represents an (integer) number≥2, especially in the range of from two to twenty, preferentially in the range of from two to ten, more preferably in the range of from three to seven;
   Y¹ represents a group R⁴OOC—CH(OH)—CH₂—C(O)— in case Z is hydrogen, or represents a group R⁴OOC—CH(OH)—CH(OH)—C(O)— in case Z is a hydroxyl group, wherein the radical R⁴ represents hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, more preferably ethyl, hydroxybutyl or hydroxypentyl, more preferably ethyl;
   Y² represents a group —O—CH(COOR⁴)—CH₂—COOR⁴ in case Z is hydrogen, or represents a group —O—CH(COOR⁴)—CH(OH)—COOR⁴ in case Z is a hydroxyl group, wherein the radical R⁴ represents, independently of one another, represents hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
however, with the proviso that at least one, especially at least two, of the radicals R³ and R⁴ represents a radical —CH(CH₃)—CH₂—C(O)OR¹, as defined above, preferentially with the (further) proviso that at least one radical R⁴ represents a radical —CH(CH₃)—CH₂—C(O)OR¹, as defined above.

Again, another object of the present invention according to a further particular embodiment is also an oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, especially an oxobutanol ester of a polymeric carboxylic acid as defined hereinabove,
wherein the oxobutanol ester corresponds to the general formula (IIIc)

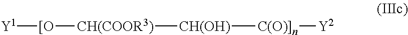

wherein, in the general formula (IIIc),
   the variable n represents an (integer) number≥2, especially in the range of from two to twenty, preferentially in the range of from two to ten, more preferably in the range of from three to seven;
   R³, independently of one another with respect to the respective repeating unit, represents: hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
   Y¹ represents a group R⁴OOC—CH(OH)—CH(OH)—C(O)—, wherein the radical R⁴ represents hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
   Y² represents a group —O—CH(COOR⁴)—CH(OH)—COOR⁴, wherein the radical R⁴, independently of one another, represents hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
however, with the proviso that at least one, especially at least two, of the radicals R³ and R⁴ represents a radical —CH(CH₃)—CH₂—C(O)OR¹, as defined above, preferentially with the (further) proviso that at least one radical R⁴ represents a radical —CH(CH₃)—CH₂—C(O)OR¹, as defined above.

Furthermore, it is also an object of the present invention according to yet another particular embodiment to provide an oxobutanol ester of a polymeric carboxylic acid, especially a 4-oxo-4-(C₁-C₅-alkoxy)-2-butanol ester or 4-oxo-4-(hydroxy-C₃-C₅-alkoxy)-2-butanol ester of a polymeric carboxylic acid, especially an oxobutanol ester of a polymeric carboxylic acid as defined hereinabove,
wherein the oxobutanol ester corresponds to the general formula (IIId)

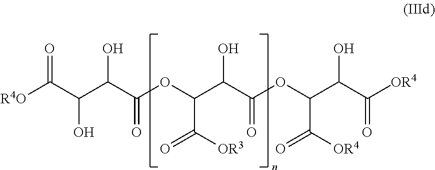

wherein, in the general formula (IIId),
   the variable n represents an (integer) number≥2, especially in the range of from two to twenty, preferentially in the range of from two to ten, more preferably in the range of from three to seven;
   R³, independently of one other with respect to the respective repeating unit, represents: hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
   R⁴, independently of one another, represents hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
however, with the proviso that at least one, especially at least two, of the radicals R³ and R⁴ represents a radical —CH(CH₃)—CH₂—C(O)OR¹, as defined above, preferentially with the (further) proviso that at least one radical R⁴ represents a radical —CH(CH₃)—CH₂—C(O)OR¹, as defined above.

The object of the present invention according to a particular embodiment is furthermore a mixture comprising at least two, especially at least three, different oxobutanol esters of a polymeric carboxylic acid, especially one or more 4-oxo-4-(C₁-C₅-alkoxy)-2-butanol esters or 4-oxo-4-(hydroxy-C₃-C₅-alkoxy)-2-butanol esters of a polymeric carboxylic acid, as defined above.

The reaction product obtainable according to the inventive method or the inventive reaction product as defined hereinabove, respectively, and/or the oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, obtainable according to the inventive production method or the inventive oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, as defined hereinabove, respectively, and/or the mixture, obtainable according to the inventive production method or the inventive mixture as defined hereinabove, respectively, comprises a multitude of advantages and special features compared to the prior art:

As the applicant has surprisingly found out, the reaction product obtainable according to the inventive method or the inventive reaction product as defined hereinabove, respectively, and/or the oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, obtainable according to the inventive production method or the inventive oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, as defined hereinabove, respectively, and/or the mixture, obtainable according to the inventive production method or the inventive mixture as defined hereinabove, respectively, is suitable as a precursor or metabolite of 3-hydroxybutyric acid or its salts, since, on the one hand, it is converted physiologically, especially in the gastrointestinal tract, to 3-hydroxybutyric acid or its salts and, on the other hand, it simultaneously comprises a good physiological compatibility or tolerability, especially with regard to non-toxicity and acceptable organoleptic properties.

Moreover, the reaction product obtainable according to the inventive method or the inventive reaction product as defined hereinabove, respectively, and/or the oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, obtainable according to the inventive production method or the inventive oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, as defined hereinabove, respectively, and/or the mixture, obtainable according to the inventive production method or the inventive mixture as defined hereinabove, respectively, is easily accessible or available on a large scale on a synthetic basis, even on a commercial scale, and with the required pharmaceutical or pharmacological quality.

Additionally, the reaction product obtainable according to the inventive method or the inventive reaction product as defined hereinabove, respectively, and/or the oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, obtainable according to the inventive production method or the inventive oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, as defined hereinabove, respectively, and/or the mixture, obtainable according to the inventive production method or the inventive mixture as defined hereinabove, respectively, can, if necessary, be provided in enantiomerically pure or enantiomerically enriched form.

The reaction product obtainable according to the inventive method or the inventive reaction product as defined hereinabove, respectively, and/or an oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, obtainable according to the inventive production method or the inventive oxobutanol ester of polymeric carboxylic acids, especially 4-oxo-4-(C₁-C₅-alkoxy)-2-butanol ester or 4-oxo-4-(hydroxy-C₃-C₅-alkoxy)-2-butanol ester of polymeric carboxylic acids, as defined hereinabove, respectively, and/or the mixture, obtainable according to the inventive production method or the inventive mixture as defined hereinabove, respectively, thus represents an efficient pharmacological drug target in the context of keto-body therapy of the human or animal body.

In the following, the remaining aspects of the invention are explained in more detail.

A further subject-matter of the present invention—according to a third aspect of the present invention—is a pharmaceutical composition, especially a drug or medicament, which comprises a reaction product obtainable according to the inventive production method or the inventive reaction product as defined hereinabove, respectively, and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method or the inventive mixture as defined hereinabove, respectively.

Especially, according to this aspect of the invention, the present invention relates to a pharmaceutical composition for the prophylactic and/or therapeutic treatment or for use in the prophylactic and/or therapeutic treatment of diseases of the human or animal body. This may especially concern diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Again, a further subject-matter of the present invention—according to a fourth aspect of the present invention—is a reaction product obtainable according to the inventive production method or the inventive reaction product as defined hereinabove, respectively, and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method or the inventive mixture as defined hereinabove, respectively, for the prophylactic and/or therapeutic treatment or for use in the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a fifth aspect of the present invention—is the use of a reaction product obtainable according to the inventive production method or the inventive reaction product as defined hereinabove, respectively, and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method or the inventive mixture as defined hereinabove, respectively, for the prophylactic and/or therapeutic treatment or for producing a pharmaceutical for the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a sixth aspect of the present invention—is the use of a reaction product obtainable according to the inventive production method or the inventive reaction product as defined hereinabove, respectively, and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method or the inventive mixture as defined hereinabove, respectively, for the prophylactic and/or therapeutic treatment or for producing a medicament for the prophylactic and/or therapeutic treatment of or for the application for catabolic metabolic states, such as hunger, diets or low-carbohydrate nutrition.

Likewise, a further subject-matter of the present invention—according to a seventh aspect of the present invention—is a food and/or a food product, which comprises a reaction product obtainable according to the inventive production method or the inventive reaction product as defined hereinabove, respectively, and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method or the inventive mixture as defined hereinabove, respectively.

According to a particular embodiment, the food and/or the food product may essentially be a dietary supplement, a functional food, a novel food, a food additive, a food supplement, a dietary food, a power snack, an appetite suppressant or a strength and/or endurance sport supplement.

Finally, yet another subject-matter of the present invention—according to an eighth aspect of the present invention—is the use of a reaction product obtainable according to the inventive production method and/or an oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, obtainable according to the inventive production method or the inventive oxobutanol ester of a polymeric carboxylic acid, especially 4-oxo-4-(C₁-C₅-alkoxy)-2-butanol ester or 4-oxo-4-(hydroxy-C₃-C₅-alkoxy)-2-butanol ester of a polymeric carboxylic acid, as defined hereinabove, respectively, and/or a mixture, obtainable according to the inventive production method, as defined hereinabove in a food and/or a food product.

According to this aspect of the invention, the food and/or the food product may especially be a dietary supplement, a functional food, a novel food, a food additive, a food supplement, a dietary food, a power snack, an appetite suppressant or a strength and/or endurance sports supplement.

Further embodiments, modifications and variations of the present invention are readily recognizable or realizable by a person skilled in the art when reading the description, without leaving the scope of the present invention.

The present invention is illustrated by the following examples, which are not intended to limit the present invention in any way, but only to explain the exemplary and non-limiting implementation and configuration of the present invention.

### EXAMPLES

### Abbreviations Used

3-BHB-EE=BHB-EE: 3-hydroxybutyric acid ethyl ester (=ethyl 3-hydroxybutanoate or 4-ethoxy-4-oxobutan-2-ol) (reactant)
EE-BHB-PWS ethyl 3-hydroxybutanoate ester of polytartaric acid
EE-BHB-PÄS ethyl 3-hydroxybutanoate ester of polymalic acid
EE-BHB-PCS ethyl 3-hydroxybutanoate ester of polycitric acid

### I. Production Examples of a Particular Embodiment of the Method According to the Invention Without the Use of a Catalyst

### I.1. Production of 4-ethoxy-4-oxobutan-2-ol esters (=ethyl 3-hydroxybutanoate esters) of Metatartaric Acid

In a 100-ml-multi-neck flask with dephlegmator (partial condenser) and distillation bridge, 11 g (R)/(S)-3-hydroxybutyric acid ethyl ester (3-BHB-EE=ethyl-3-hydroxybutanoate or 4-ethoxy-4-oxobutan-2-ol) (racemic) and 5 g metatartaric acid (polytartaric acid) are provided. The reaction mixture is reacted with stirring at 120° C. for 6 h and the resulting reaction water is continuously removed by distillation. A sample of the reaction mixture for further analysis is taken after 3 h and after 6 h. The reaction water is removed by distillation. Subsequently, the excess 3-hydroxybutyric acid ethyl ester is evaporated in a nitrogen stream at 70° C., and then another sample is taken. The residue obtained is steam treated for 2 to 4 h in high vacuum.

A mixture of ethyl 3-hydroxybutanoate mono-, di- and higher esters of metatartaric acid is obtained (i.e. mixture of metatartaric acid mono(4-ethoxy-4-oxo-butan-2-ol) ester, metatartaric acid di(4-ethoxy-4-oxo-butan-2-ol) ester and metatartaric acid poly(4-ethoxy-4-oxo-butan-2-ol) ester).

Characterization is performed by gel permeation chromatography (GPC) and acid number determination.

The acid number (SZ) indicates the mass of potassium hydroxide (KOH) in milligrams (mg) required to neutralize one gram of the acid to be analyzed (in this case, metatartaric acid). The acid number is a measure of the carboxylic acid groups in a chemical compound and is used to quantify the acidity of a substance.

The acid number of the pure metatartaric acid (reactant) is 469 mg KOH/g and the acid number of the obtained reaction product is 190 mg KOH/g. Thus, the number of free carboxylic acid groups has decreased significantly, thus a successful esterification with ethyl 3-hydroxybutyric acid ester has taken place.

Samples taken during the synthesis (after 3 h of reaction time as well as after 6 h of reaction time and after nitrogen evaporation) and pure metatartaric acid (reactant) are analyzed by gel permeation chromatography (GPC); GC area analyses are summarized in Table 1:

TABLE 1
GC area analysis [%] of the reaction of metatartaric
acid with ethyl 3-hydroxybutanoate

| Metatartaric acid | | EE-BHB-PWS | | EE-BHB-PWS | | EE-BHB-PWS | |
|---|---|---|---|---|---|---|---|
| M | GPC area | M | GPC area | M | GPC area | M | GPC area |
| [g/mol] | [%] | [g/mol] | [%] | [g/mol] | [%] | [g/mol] | [%] |
| 150 * | 3.69 | 150 * | 2.36 | 150 * | 2.05 | 150 * | 0.59 |
| — | — | 190 ** | 27.43 | 190 ** | 18.81 | 190 ** | 3.17 |
| — | — | — | — | 230 | 3.05 | 230 | 3.21 |
| 360 | 23.6 | 340 | 11.08 | 320 | 12.33 | 320 | 13.34 |
| — | — | 440 | 5.64 | 440 | 7.49 | 440 | 10.13 |
| 570 | 17.33 | 550 | 5.96 | 550 | 6.13 | 550 | 7.42 |
| 740 | 14.17 | 690 | 7.04 | 690 | 7.26 | 690 | 8.59 |
| 890 | 14.76 | 850 | 6.6 | 850 | 6.67 | 850 | 8.14 |
| 960 | 24.97 | 1000 | 33.58 | 1000 | 36.21 | 990 | 45.41 |
| Mw/Mn | 710/530 | | 890/390 | | 980/430 | | 1200/610 |
| PDI | 1.34 | | 2.28 | | 2.28 | | 1.97 |

* tartaric acid
** 3-BHB-EE

The reaction course is shown schematically below:

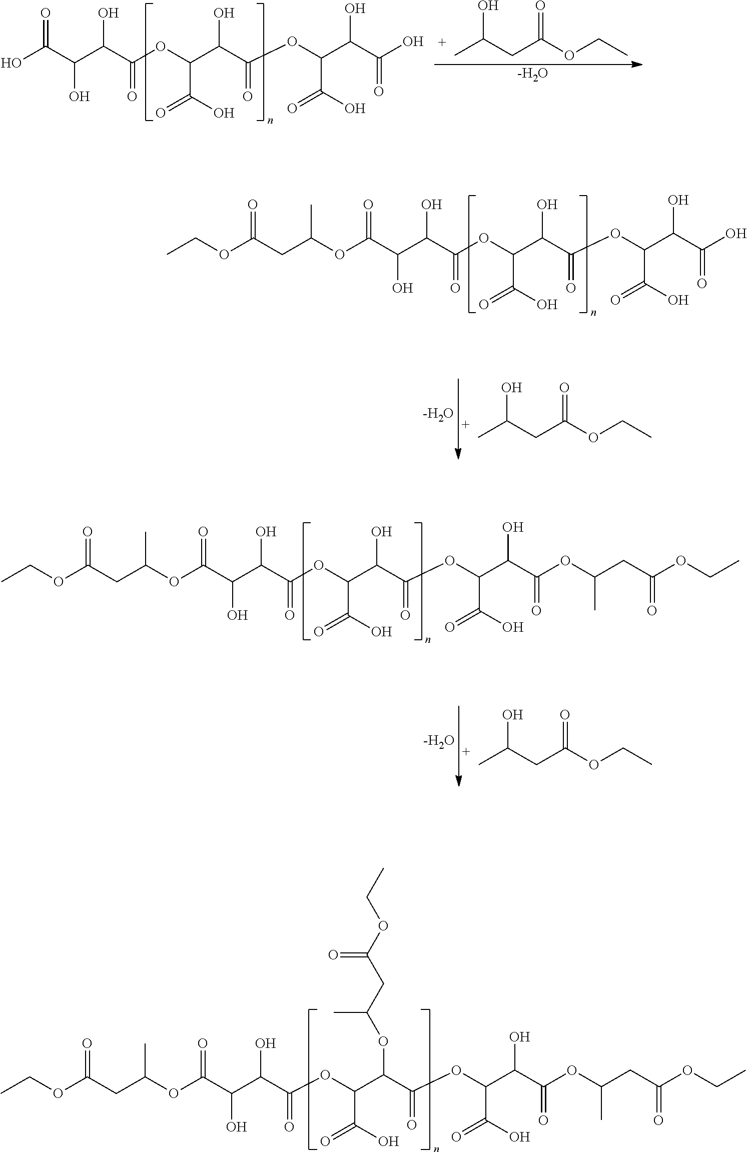

### I.2. Production of 4-ethoxy-4-oxobutan-2-ol esters (=ethyl 3-hydroxybutanoate esters) of Polymalic Acid

In a 100-ml-multi-neck flask with dephlegmator (partial condenser) and distillation bridge, 11 g (R)/(S)-3-hydroxybutyric acid ethyl ester (3-BHB-EE=ethyl-3-hydroxybutanoate or 4-ethoxy-4-oxobutan-2-ol) (racemic) and 5 g polymalic acid are provided. The reaction mixture is reacted with stirring at 120° C. for 6 h and the resulting reaction water is continuously removed by distillation. Subsequently, the excess 3-hydroxybutyric acid ethyl ester is evaporated in a nitrogen stream at 70° C. and then another sample is taken. The residue obtained is steam treated for 2 to 4 h in high vacuum.

A mixture of ethyl 3-hydroxybutanoate mono-, di- and higher esters of polymalic acid is obtained.

Characterization is performed by gel permeation chromatography (GPC) and acid number determination.

### I.3. Production of 4-ethoxy-4-oxobutan-2-ol esters (=ethyl 3-hydroxybutanoate esters) of Polycitric Acid

In a 100-ml-multi-neck flask with dephlegmator (partial condenser) and distillation bridge, 11 g (R)/(S)-3-hydroxybutyric acid ethyl ester (3-BHB-EE=ethyl 3-hydroxybutanoate or 4-ethoxy-4-oxobutan-2-ol) (racemic) and 5 g polycitric acid are provided. The reaction mixture is reacted with stirring at 120° C. for 6 h and the resulting reaction water is continuously removed by distillation. Subsequently, the excess 3-hydroxybutyric acid ethyl ester is evaporated in a nitrogen stream at 70° C. and then another sample is taken. The residue obtained is steam treated for 2 to 4 h in high vacuum.

A mixture of ethyl 3-hydroxybutanoate mono-, di- and higher esters of polycitric acid is obtained.

Characterization is performed by gel permeation chromatography (GPC) and acid number determination.

### I.4. Production of Further 4-ethoxy-4-oxobutan-2-ol esters (=ethyl 3-hydroxybutanoate esters) of Polymeric Carboxylic Acids

In addition, the above syntheses Nos. I.1, I.2 and I.3 are each repeated, however each with different 3-hydroxybutanoates (namely in each case 3-hydroxybutyric acid (hydroxybutyl) ester or 3-hydroxybutyric acid (hydroxypentyl) ester instead of 3-hydroxybutyric acid ethyl ester). 3-Hydroxybutyric acid (hydroxybutyl) ester is obtained by esterification of 3-hydroxybutyric acid with butanediol (e.g. with 1,3-butanediol), while 3-hydroxybutyric acid (hydroxypentyl) ester is obtained by esterification of 3-hydroxybutyric acid with pentanediol (e.g. with 1,3-pentanediol). Comparable results are obtained. Purification and separation are carried out in the same way.

Also, the previously mentioned production examples are repeated, however with the methyl and ethyl esters of the polymeric carboxylic acids. Comparable results are obtained. Purification and separation are carried out in the same way.

### II. Production Examples of an Alternative Particular Embodiment of the Method According to the Invention Using a Metal Catalyst

All chemical synthesis examples described previously in section I. are carried out again, however with the addition of titanium tetrabutylate as a catalyst (titanium(IV)-catalyst). The titanium(IV)-catalyst is provided in the flask together with the other reactants. Subsequently, the reaction method corresponds to the examples described above. Comparable results are obtained. The catalyst is separated and recycled at the end of the reaction.

### III. Production Examples of a Further Alternative Particular Embodiment of the Method According to the Invention Using an Enzyme Catalyst

All chemical synthesis examples previously described in section I. are carried out again, however with the addition of an immobilized enzyme (CALB lipase on polymer support, derived from Candida antarctica, e.g. Novozym® 435 from Sigma-Aldrich or Merck or Lipozym® 435 from Strem Chemicals, Inc.) as a catalyst and at 70° C. under vacuum. Comparable results are obtained. The catalyst (i.e. the enzyme) is separated and recycled after the end of the reaction.

### IV. Physiological Application Tests: In Vitro Digestion Tests

### Digestion Experiments (Splitting or Cleavage Experiments) of Inventive 3-hydroxybutanoate esters of Polymeric Carboxylic Acids

By means of cleavage experiments it is shown that 3-hydroxybutanoate esters of polymeric carboxylic acids or mixtures thereof prepared according to the invention (cf. previously described experiments according to I., II. and III.), including reaction by-products, can be cleaved in the human gastrointestinal tract.

In each case, purified reaction products obtained by the method of the invention (i.e. 3-hydroxybutanoate ester of polytartaric acid, 3-hydroxybutanoate ester of polymalic acid, and 3-hydroxybutanoate ester of polycitric acid) are used as starting mixtures.

For the cleavage experiments under near-body conditions two media are investigated:
FaSSGF, which simulates the stomach
FaSSIF, which simulates the intestinal tract

Both media are from the company Biorelevant®, Ltd. in Great Britain. In addition, in some experiments porcine pancreas is added (Panzytrat® 40,000, Fa. Allergan).

The results of the cleavage experiments in a FaSSGF or FaSSIF medium with Panzytrat® and without Panzytrat® (both 35° C., 24 h) show that the samples hydrolyze under FaSSGF conditions with Panzytrat® and without Panzytrat®; this is mainly due to the low pH value (pH=1.6) of the medium. Under FaSSIF conditions, a lower conversion using Panzytrat® takes place.

In the cleavage experiments, it can be seen that the cleavage of 3-hydroxybutanoate proceeds in a cascade (i.e. that the 3-hydroxybutanoate is released stepwise and can subsequently be further cleaved to the free 3-hydroxybutyric acid and ethanol).

### Further Digestion Experiments (Cleavage Experiments) of Inventive Carboxylic Acids of 3-hydroxybutanoate

### Cleavage Experiments With Pancreatin

2 g of a 3-hydroxybutanoate ester of a polymeric carboxylic acid prepared as described above or a corresponding mixture is dissolved in 50 g water and 0.5 g (1% by weight) pancreatin is added. The pancreatin is used in the form of the commercially available product Panzytrat® 40,000 from the Allergan company. The whole mixture is stirred on a hot plate at 50° C.; the course of the reaction is determined and monitored by continuously recording the acid number over time. The acid number increases over the observation period (cleavage of the 3-hydroxybutanoate esters of polymeric carboxylic acids to the free 3-hydroxybutanoate). The conversion-time-course of the aqueous cleavage of the 3-hydroxybutanoate esters of polymeric carboxylic acids according to the invention by pancreatin, including the increase of the acid number over time, demonstrates the desired decomposition of the reactant mixture to the free polymeric carboxylic acid and the 3-hydroxybutanoate. This is confirmed by corresponding analytics. The experiment proves that the starting mixture according to the invention is a suitable physiological precursor for 3-hydroxybutyric acid or its esters (3-hydroxybutanoates) for the corresponding keto-body therapies.

The experiment is carried out and verified in each case using the individual esters in pure form. Comparable results are obtained in each case, i.e. the 3-hydroxybutanoate esters of polymeric carboxylic acids are each cleaved by pancreatin to give the free polymeric carboxylic acid and 3-hydroxybutanoate.

The cleavage experiments described above demonstrate that the 3-hydroxybutanoate esters of polymeric carboxylic acids are efficient precursors or metabolites of free hydroxybutyric acid or its esters (here: ethyl esters, hydroxybutyl esters and hydroxypentyl esters), especially with regard to their intended effect, which are present in physiologically compatible or physiologically compatible form. Likewise, metabolically usable or convertible polymeric carboxylic acids based on carboxylic acids occurring in the natural metabolism (e.g. citrate cycle) are also formed (e.g. polycitric acid or polycitrates, polymalic acid or polymalates, polytartaric acid or metatartaric acid or polytartrates etc.).

### VI. Further Testing (Organoleptic and Toxicity)

Further experiments and test series are carried out with respect to organoleptic and toxicity of the 3-hydroxybutanoate esters of polymeric carboxylic acids according to the invention. These show that the inventive 3-hydroxybutanoate esters of polymeric carboxylic acids are organoleptically acceptable and compatible, especially exhibit significantly improved organoleptic properties compared to pure 3-hydroxybutyric acid as well as its salts and esters, and furthermore do not exhibit any toxicity that would conflict with the application.

1-56. (canceled)

57. A method for producing oxobutanol esters of polymeric carboxylic acids,
wherein at least one oxobutanol of the general formula (I) 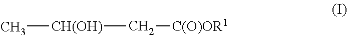
wherein, in the general formula (I), the radical R¹ is selected among C₁-C₅-alkyl and hydroxy-C₃-C₅-alkyl,
is reacted, in an esterification reaction, with at least one polymeric carboxylic acid (II) comprising at least three carboxyl groups, wherein the at least one polymeric carboxylic acid (II) is selected from the group consisting of polytartaric acid, polymalic acid and polycitric acid and their salts, partial salts, esters and partial esters as well as combinations and mixtures thereof,
so that, as a reaction product (III), at least one oxobutanol ester of the polymeric carboxylic acid (II) is obtained.

58. The method according to claim 57,
wherein the reaction is carried out in the absence of any solvent.

59. The method according to claim 57,
wherein the reaction is carried out in the absence of any catalyst;
wherein the reaction is carried out at temperatures in the range of from 20° C. to 160° C. and at a pressure in the range of from 0.0001 bar to 10 bar, and
wherein the reaction is carried out in the presence of an inert gas selected from the group consisting of helium, argon or nitrogen, preferably in the presence of nitrogen.

60. The method according to claim 57,
wherein the reaction is carried out in the presence of an enzyme as a catalyst;
wherein the enzyme is selected from the group consisting of synthetases, catalases, esterases, lipases and combinations thereof,
wherein the enzyme is recycled after the reaction,
wherein the reaction is carried out at temperatures in the range of from 10° C. to 80° C. and at a pressure in the range of from 0.0001 bar to 10 bar.

61. The method according to claim 57,
wherein the reaction is carried out in the presence of a metal-containing acidic or basic catalyst;
wherein the catalyst is selected from the group consisting of (i) basic catalysts selected among alkali or alkaline earth hydroxides and alkali or alkaline earth alcoholates, (ii) acidic catalysts selected among mineral acids and organic acids, (iii) Lewis acids based on titanium, tin, zinc and aluminum compounds, and (iv) heterogeneous catalysts based on mineral silicates, germanates, carbonates and aluminum oxides, as well as combinations thereof,
wherein the catalyst is recycled after the reaction, and
wherein the reaction is carried out at temperatures in the range of from 20° C. to 160° C. and at a pressure in the range of from 0.0001 bar to 10 bar.

62. The method according to claim 57,
wherein the oxobutanol of the general formula (I) and the polymeric carboxylic acid (II) are used in a molar ratio of oxobutanol of the general formula (I)/carboxyl groups of the polymeric carboxylic acid (II) in a range of from 1:1 to 10:1.

63. The method according to claim 57,
wherein hydroxyl groups and carboxyl groups still present in the reaction product after the reaction has been performed are at least partially functionalized.

64. The method according to claim 57,
wherein hydroxyl groups and carboxyl groups still present in the reaction product after the reaction has been performed are at least partially functionalized via esterification.

65. A reaction product in the form of an oxobutanol ester of a polymeric carboxylic acid or a mixture of several oxobutanol esters of a polymeric carboxylic acid,
wherein the reaction product is obtainable by the method according to claim 57.

66. An oxobutanol ester of a polymeric carboxylic acid,
wherein the polymeric carboxylic acid is selected from the group consisting of polytartaric acid, polymalic acid and polycitric acid and their salts, partial salts, esters and partial esters as well as combinations and mixtures thereof; and
wherein at least one carboxyl group of the polymeric carboxylic acid is esterified with an oxobutanol of the general formula (I) 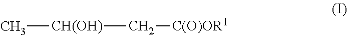
wherein, in the general formula (I), the radical R¹ is selected among C₁-C₅-alkyl and hydroxy-C₃-C₅-alkyl.

67. The oxobutanol ester of a polymeric carboxylic acid according to claim 66,
wherein the at least one carboxyl group of the polymeric carboxylic acid which is esterified with an oxobutanol of the general formula (I) is at least one terminal carboxyl group, and
wherein, in the general formula (I), the radical R¹ is selected among ethyl, butyl, pentyl, hydroxybutyl and hydroxypentyl.

68. The oxobutanol ester of a polymeric carboxylic acid according to claim 66,
wherein the polymeric carboxylic acid corresponds to the general formula (IId) 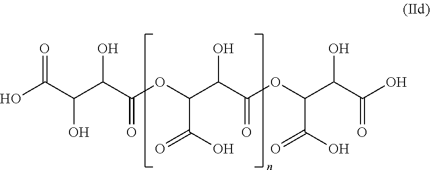
wherein, in the general formula (IId), the variable n represents an integer number≥2.

69. The oxobutanol ester of a polymeric carboxylic acid according to claim 66,
wherein the polymeric carboxylic acid corresponds to the general formula (IId) 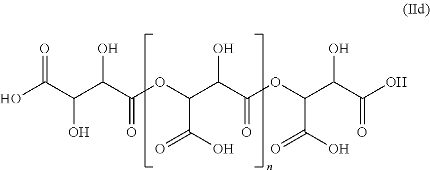
wherein, in the general formula (IId), the variable n represents an integer number in the range of from 2 to 20.

70. The oxobutanol ester of a polymeric carboxylic acid according to claim 66,
wherein the polymeric carboxylic acid corresponds to the general formula (IId) 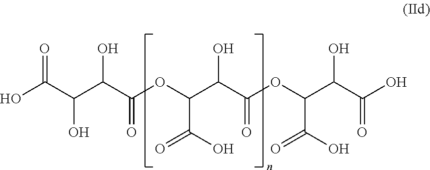
wherein, in the general formula (IId), the variable n represents an integer number in the range of from 3 to 7.

71. The oxobutanol ester of a polymeric carboxylic acid according to claim 66,
wherein the oxobutanol ester corresponds to the general formula (IIId) 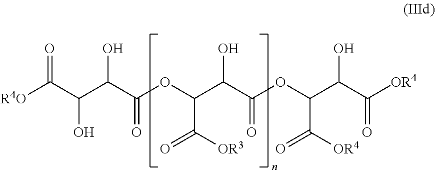
wherein, in the general formula (IIId),
the variable n represents an integer number≥2;
R³, independently of one other with respect to the respective repeating unit, represents: hydrogen or a radical —CH(CH₃)—CH₂—C(O)OR¹, wherein the radical R¹ represents C₁-C₅-alkyl or hydroxy-C₃-C₅-alkyl;
R⁴, independently of one another, represents hydrogen or a radical —CH(CH₃)—CH₂—C(O)OR¹, wherein the radical R¹ represents C₁-C₅-alkyl or hydroxy-C₃-C₅-alkyl;
however, with the proviso that at least one of the radicals R³ and R⁴ represents a radical —CH(CH₃)—CH₂—C(O)OR¹, wherein the radical R¹ represents C₁-C₅-alkyl or hydroxy-C₃-C₅-alkyl.

72. A mixture comprising at least two different oxobutanol esters of a polymeric carboxylic acid, as defined in claim 66.

73. A pharmaceutical composition comprising at least one oxobutanol ester of a polymeric carboxylic acid,
wherein the polymeric carboxylic acid is selected from the group consisting of polytartaric acid, polymalic acid and polycitric acid and their salts, partial salts, esters and partial esters as well as combinations and mixtures thereof; and
wherein at least one carboxyl group of the polymeric carboxylic acid is esterified with an oxobutanol of the general formula (I) 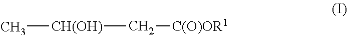
wherein, in the general formula (I), the radical R¹ is selected among C₁-C₅-alkyl and hydroxy-C₃-C₅-alkyl.

74. The pharmaceutical composition according to claim 73,
wherein the polymeric carboxylic acid corresponds to the general formula (IId) 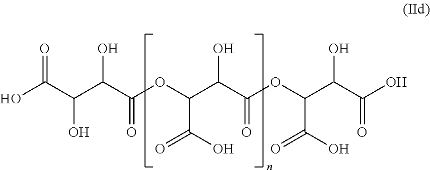
wherein, in the general formula (IId), the variable n represents an integer number≥2.

75. The pharmaceutical composition according to claim 73,
wherein the oxobutanol ester corresponds to the general formula (IIId) 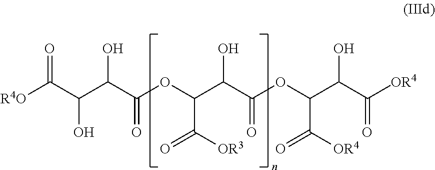
wherein, in the general formula (IIId),
the variable n represents an integer number≥2;
R³, independently of one other with respect to the respective repeating unit, represents: hydrogen or a radical —CH(CH₃)—CH₂—C(O)OR¹, wherein the radical R¹ represents C₁-C₅-alkyl or hydroxy-C₃-C₅-alkyl;
R⁴, independently of one another, represents hydrogen or a radical —CH(CH₃)—CH₂—C(O)OR¹, wherein the radical R¹ represents C₁-C₅-alkyl or hydroxy-C₃-C₅-alkyl;
however, with the proviso that at least one of the radicals R³ and R⁴ represents a radical —CH(CH₃)—CH₂—C(O)OR¹, wherein the radical R¹ represents C₁-C₅-alkyl or hydroxy-C₃-C₅-alkyl.

76. The pharmaceutical composition according to claim 73,
wherein the pharmaceutical composition is a drug or a medicament.

77. A method of treating a human or an animal suffering from a disease of the human or animal body,
wherein the method comprises the step of administering to said human or animal a therapeutically efficient amount of at least one oxobutanol ester of a polymeric carboxylic acid,
wherein the polymeric carboxylic acid is selected from the group consisting of polytartaric acid, polymalic acid and polycitric acid and their salts, partial salts, esters and partial esters as well as combinations and mixtures thereof; and
wherein at least one carboxyl group of the polymeric carboxylic acid is esterified with an oxobutanol of the general formula (I) 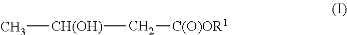
wherein, in the general formula (I), the radical R¹ is selected among C₁-C₅-alkyl and hydroxy-C₃-C₅-alkyl.

78. The method according to claim 77,
wherein the disease is selected among diseases associated with a disorder of human or animal energy metabolism.

79. The method according to claim 77,
wherein the disease is selected among diseases associated with a disorder of human or animal keto-body metabolism.

80. The method according to claim 77,
wherein the disease is selected among craniocerebral trauma, stroke, hypoxia, cardiovascular diseases, myocardial infarction, refeeding syndrome, anorexia, epilepsy, neurodegenerative diseases, dementia, Alzheimer's disease, Parkinson's disease, multiple sclerosis, amyotrophic lateral sclerosis, fat metabolic diseases, glucose transporter defect, GLUT1 defect, very long-chain acyl-CoA dehydrogenase deficiency, mitochondriopathies, mitochondrial thiolase defect, Huntington's disease, cancers, T-cell lymphomas, astrocytomas, glioblastomas, HIV, rheumatic diseases, rheumatoid arthritis, arthritis urica, diseases of the gastrointestinal tract, chronic inflammatory bowel diseases, ulcerative colitis, Crohn's disease, lyosomal storage diseases, sphingolipidosis, Niemann-Pick disease, diabetes mellitus and chemotherapy-caused side-effects.

81. A food product comprising at least one oxobutanol ester of a polymeric carboxylic acid,
wherein the polymeric carboxylic acid is selected from the group consisting of polytartaric acid, polymalic acid and polycitric acid and their salts, partial salts, esters and partial esters as well as combinations and mixtures thereof; and
wherein at least one carboxyl group of the polymeric carboxylic acid is esterified with an oxobutanol of the general formula (I) 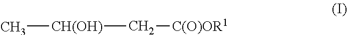
wherein, in the general formula (I), the radical R¹ is selected among C₁-C₅-alkyl and hydroxy-C₃-C₅-alkyl.

82. The food product according to claim 81,
wherein the food product is selected from the group consisting of a food, a dietary supplement, a functional food, a novel food, a food additive, a food supplement, a dietary food, a power snack, an appetite suppressant and a strength sports supplement and an endurance sports supplement.
